(19) United States
(12) Patent Application Publication
(10) Pub. No.: US 2025/0197813 A1
(43) Pub. Date: Jun. 19, 2025

(54) A METHOD FOR MANUFACTURING A PERFUSABLE THREE-DIMENSIONAL TISSUE MODEL WITH 3D BIOPRINTING TECHNOLOGY, AND A TISSUE MODEL PRODUCED WITH THIS METHOD

(71) Applicant: POLBIONICA SP. Z O.O., Warszawa (PL)

(72) Inventors: Michal Wszola, Warszawa (PL); Marta Klak, Pila (PL); Andrzej Berman, Warszawa (PL); Sylwester Domanski, Warszawa (PL); Tomasz Dobrzanski, Warszawa (PL); Michal Rachalewski, Jozefoslaw (PL); Oliwia Janowska, Jastrzab (PL)

(21) Appl. No.: 18/847,642

(22) PCT Filed: Mar. 17, 2023
(86) PCT No.: PCT/PL2023/050019

§ 371 Date: Sep. 16, 2024

(30) Foreign Application Priority Data
Mar. 17, 2022 (EP) 22461525.2

(51) Int. Cl. C12N 5/071; B33Y 10/00; B33Y 70/00; B33Y 80/00

(52) U.S. Cl. CPC C12N 5/0697; B33Y 10/00; B33Y 70/00; B33Y 80/00; C12N 2502/1323; C12N 2502/28; C12N 2513/00; C12N 2533/54; C12N 2533/80; C12N 2537/10; C12N 2539/00

(57) ABSTRACT

A method For manuFacturing a perFusable three-dimensional tissue model, containing therein a channel distributed across its entire structure, enabling the Flow oF Fluids, wherein bioprinting a vascular system with the extrusive method using bioink, the walls opening and closing the channel in its upper part being printed parallel to the channel axis, and bioprinting oF the model body with the extrusive method using bioink, the bioink For printing the body being diFFerent From the bioink used For bioprinting the vessels, placing the resulting system in an incubator, in a temperature in which bioink For printing the vascular system undergoes melt, removing the bioink, optionally, causing growth in the channel by means oF cells in a medium, wherein, the cross-section oF the channel being the same as the cross-section oF native vessels present in a living organism. The inventions relates to a bionic model with a perFusable system.

The object of the invention is a method for manufacturing a perFusable three-dimensional tissue model with 3D bioprinting technology. The object of the invention is also a tissue model produced with this method. The invention is found useful in the Field of bioprinting bionic organs.

The document US20210041853A1 describes methods of vascular network models printed by means of 3D printing, including narrowed pulmonary arteries, capable of vascular perfusion. The described method involves acquiring an image of the copied anatomy, and subsequently generating a geometric tissue model based on this image. Therefore, the disclosed solution focuses on the method of generating a 3D model of the copied source material, but does not provide information about subjects related to the printing method or the type of the applied bioink.

The document US20200316254A1 describes bioprinting of a cardiac patch with perfusive architecture. It comprises perFusable vessels embedded integratedly between two layers of anisotropically oriented myocardial fibres. The cardiac patch is made using a dual 3D bioprinting technique using stereolithography to form an anisotropic construct, and extrusion printing to form perfusion vessels. The applied bioink contains, inter alia, gelatin methacrylate (GelMA) and polyethylene glycol diacrylate (PEGDA). The anisotropically oriented myocardial fibres are formed in layers, and the perFusable vessels are formed by at least every fifth layer of anisotropically oriented myocardial fibres.

The document KR101974716B1 describes 3D printing of blood vessels. The tissues are printed in such a way that they consist of two layers, wherein bioinks of various compositions can be printed on the layers.

The document US20200047399A1 describes a method of 3D printing of soFt polymer material. The method uses extrusion printing of polymer solutions, and it requires sequential printing of a solution of a photocurable polymer.

The document US20200024560A1 describes bioprinting of myocardial tissue. The 3D printing method described in the document comprises, inter alia, producing hydrogel scaffolds made of microfibres, and bioprinting endothelial cells directly in the scaffolds, the step of bioprinting taking place simultaneously with the step of producing the microfibres. Bioprinting is done with an alginate-gelatin-methacrylate mixture (GelMA), and the printing process takes place with controlled anisotropy. The produced hydrogel scaffold may consist of numerous layers, each layer being stacked in a crossed configuration, so that the main axes of the consecutive layers are perpendicular.

The document WO2019237061A1 discloses a method of producing a biologically printable 3D platform. The applied hydrogel contains gelatin methacrylate, collagen and fibrins, and it also contains cells, the hydrogel creating a three-dimensional tissue structure around the three-dimensional network of hollow vascular channels.

The document CN112891633A describes producing a vascularised musculocutaneous flap by means of 3D bioprinting. A mixture of gelatin with methacrylic anhydride, sodium alginate, polyethylene glycol diacrylate, and the cells of vascular smooth muscles is used as bioink. The printed scaffold is subsequently crosslinked by blue light.

The document EP3655052A4 describes a method for producing a perfusional multi-layered tissue model, comprising depositing one or more fibres on a substrate, each of which contains a plurality of concentric and coaxial layers saturated with cells and extending over at least a partial length of the Fibre. The result of bioprinting is a perfusional multi-layered construct of tubular tissue.

From prior art there are no known solutions describing the bioprinting of a model which has a channel with a cross-section matching the cross-section of native vessels present in a living organism. Moreover, there are no solutions in which walls closing a channel would be printed according to a pattern, in which the Fibres are printed parallel to the channel axis (FIG. 1B), which would eliminate the risk of coagulation inside a bionic organ.

The purpose of the invention is to develop a method for manufacturing a tissue model by means of 3D printing, which would eliminate the risk of the creation of cloth inside the produced model. In this method, the Fibres are printed parallel to the axis of the channel present inside the model.

The subject of the invention is a method for manufacturing a perFusable three-dimensional tissue model, containing therein a channel distributed across its entire structure, enabling the Flow of fluids, which comprises:
i) Bioprinting a vascular system with the extrusive method using bioink, the walls opening and closing the channel in its upper part being printed parallel to the channel axis, and bioprinting of the model substance with the extrusive method using bioink, the bioink for printing the body being diFFerent From the bioink used for bioprinting the vessels,
ii) placing the resulting system in an incubator, in a temperature in which bioink for printing the vascular system undergoes melt,
iii) removing the bioink,
iv) optionally, causing growth in the channel by means of cells in a medium,
the cross-section of the channel being the same as the cross-section of native vessels present in a living organism.

Preferably, the temperature of the print head with bioink ranges from 10 to 26° C., the printing needle has a diameter ranging from 100 to 609 nm, the pressure used in the bioprinting process encompasses a range of 5 to 200 kPa, and the Fibre printing rate ranges from 5 to 40 mm/s, the length of an individual printed fibre ranging from 150 mm to 5000 mm.

Preferably, the pressure used in the process of bioprinting the body of the model and the layers surrounding the channel ranges from 5 to 40 kPa in the case of printing by means of bioink containing pancreatic islets, and from 5 to 200 kPa in the case of printing by means of bioink containing cells, in particular endothelial cells or fibroblasts.

Preferably, the pressure used in the process of bioprinting a vascular system ranges from 5 to 200 kPa in the case of printing by means of bioink containing cells, in particular endothelial cells or fibroblasts, and at least 5 kPa in the case of printing by means of bioink not containing cells.

Preferably, at least one print head with temperature control is used for printing.

Preferably, bioink for printing the vessels is hydrogel containing an extracellular matrix, with a concentration of 5 to 10% in a solution of phosphate-buffered physiological saline, produced via sonification.

Preferably, bioink for printing the vessels is a nonionic copolymer surfactant, preferably a compound with the Formula (C₃H₆O·C₂H₄O)ₓ, x standing for 10 to 1000 repetitions.

Preferably, the bioink used for printing vessels is a suspension containing endothelial cells and fibroblasts in a ratio of 1:2, and with a total number of cells of 5 to 10 million/ml, preferably with the number of cells being 8 million/ml in hydrogel containing an extracellular matrix with a concentration of 5 to 10% in a solution of phosphate-buffered physiological saline.

Preferably, the bioink used for printing the body is a solution of a decellularized extracellular matrix treated with an enzyme, preferably pepsin, which contains at least one crosslinking agent and a photoinitiator; preferably, the crosslinking agent is gelatin methacrylate, gelatin methacrylamide and/or hyaluronic acid methacrylate, and the photoinitiator is lithium phenyl-2,4,6-trimethylbenzoylphosphinate.

Preferably, step ii) takes place in a temperature of no more than 37° C., over a time of no more than 24 h, preferably over a time oF 30 to 40 min.

Preferably, the removal of bioink in step iii) takes place by rinsing the channel with a solution of phosphate-buffered physiological saline, a cell medium or another fluid with properly adjusted temperature, in which bioink undergoes melt.

Preferably, the cells for growing in step iv) are selected from a group of: endothelial cells, fibroblasts or a mixture thereof in proportions of 1:2; preferably, the cells grown in the channel have a concentration of 5 to 10 million/ml, preferably 8 million/ml in a culture medium.

The invention also relates to a bionic model with a perFusable system, in which:
i) the model body is printed by means of bioink constituting a solution of a decellularized extracellular matrix treated with pepsin, which contains at least one crosslinking agent and a photoinitiator; preferably, the crosslinking agent is gelatin methacrylate, gelatin methacrylamide and/or hyaluronic acid methacrylate, and the photoinitiator is lithium phenyl-2,4,6-trimethylbenzoylphosphinate;
ii) the channel filling is printed by means of hydrogel containing a decellularized extracellular matrix, which optionally contains endothelial cells and fibroblasts in a ratio of 1:2, and with a total number of cells of 5 to 10 million/ml, with a concentration of 5 to 10% in a solution of phosphate-buffered physiological saline; this hydrogel is produced by sonification, or
the channel filling is printed by means of a nonionic copolymer surfactant.

Preferably, there are 2 to 4 outline layers for the bionic channel of the model per each model body filling layer, the pathways of the consecutive layers being arranged at an angle up to and including 90° relative to each other, and the layers surrounding the channel are printed parallel to each other, in the same direction.

Preferably, the layers opening and closing the model body, and the layers opening and closing the vascular channel are printed in a number of 1 to 10 layers; each layer has a height of 0.1 to 1.2 mm; the layers of the pancreas body are printed in a sandwich system with a ratio of the height of the layers to the height of the channel outline wall ranging from 1:1.1 to 1:2.

Preferably, the bionic model has a channel distributed across its entire structure, the cross-section of the channel being the same as the cross-section of native vessels present in a living organism.

The object of the invention is presented in the drawing, in which:

FIG. 1A presents the printing pattern of a vascular system—a version with printing transverse to the channels oF the model.

FIG. 1B presents the printing pattern of a vascular system—a version with printing along the channels of the model.

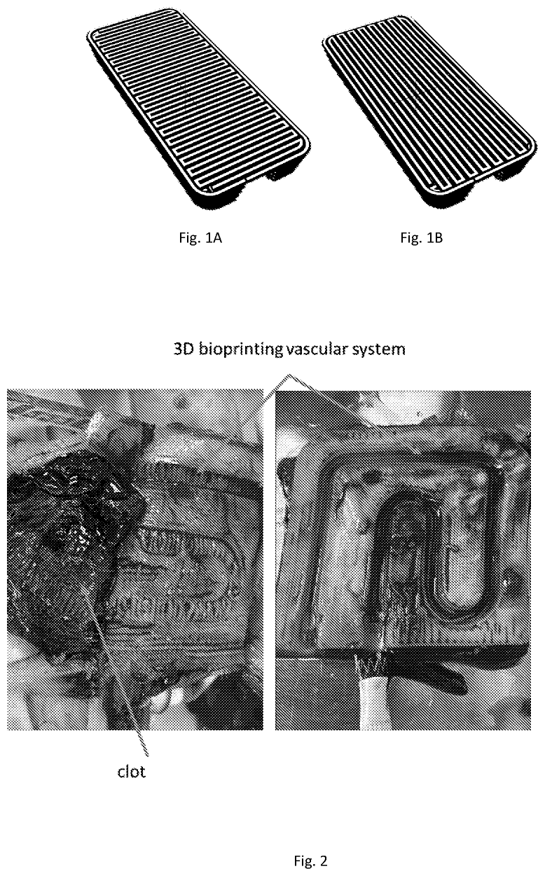

FIG. 2 presents an image of thrombotic tissue visible after resection of the bionic pancreas. The cloth were caused by the method of printing the vascularisation of the bionic pancreas. Changing the printing direction to lengthwise has eliminated this problem (the right-hand image).

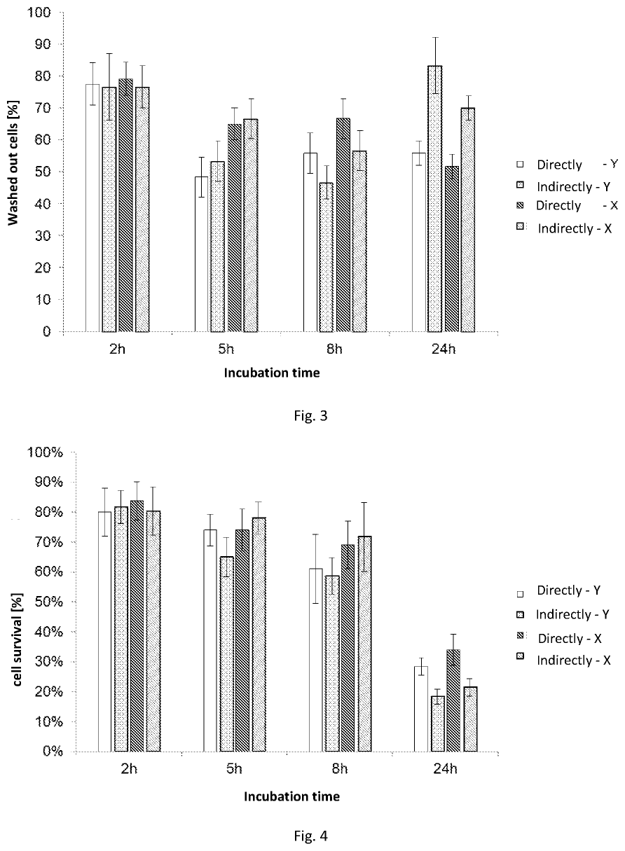

FIG. 3. The number of washed out cells expressed in percentage points for the tested options of incubation time in a bioreactor/incubator, taking into account the printing technique (channel printing in the X and Y axes) and the cell introduction method (indirectly, using the so-called vascular bioink; directly, after washing out Pluronic). The error bars correspond to standard deviations.

FIG. 4. The longevity of washed out cells expressed in percentage points for the tested options of incubation time in a bioreactor/incubator, taking into account the printing technique (channel printing 20 in the X and Y axes) and the cell introduction method (indirectly, using the so-called vascular bioink; directly, after washing out Pluronic). The error bars correspond to standard deviations.

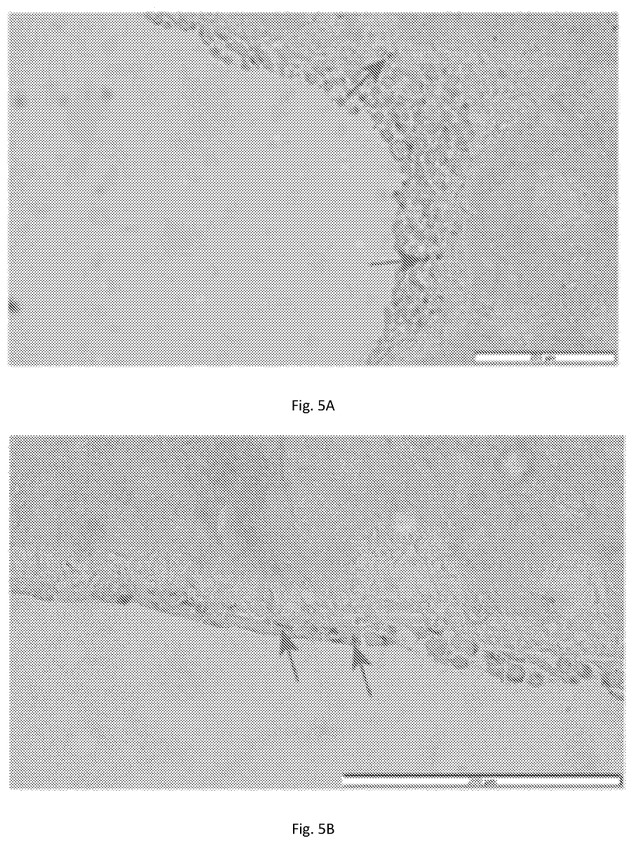

FIG. 5. Channel scraps in a cross-section, stained with haematoxylin (FIG. 5A) and eosin (FIG. 5B). The arrows indicate the cell nuclei of the cells adjacent to the channel walls.

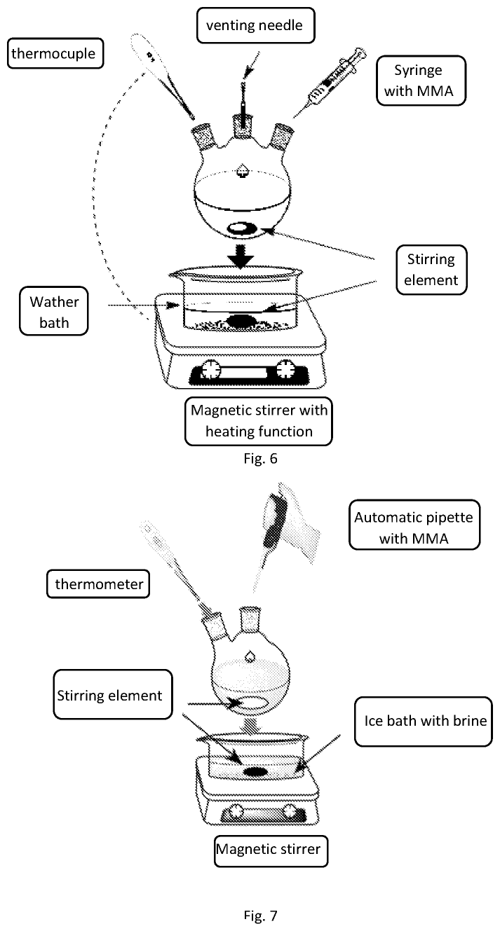

FIG. 6 presents a reaction mechanism for performing methacrylation of gelatin.

FIG. 7 presents a reaction mechanism for performing methacrylation of hyaluronic acid.

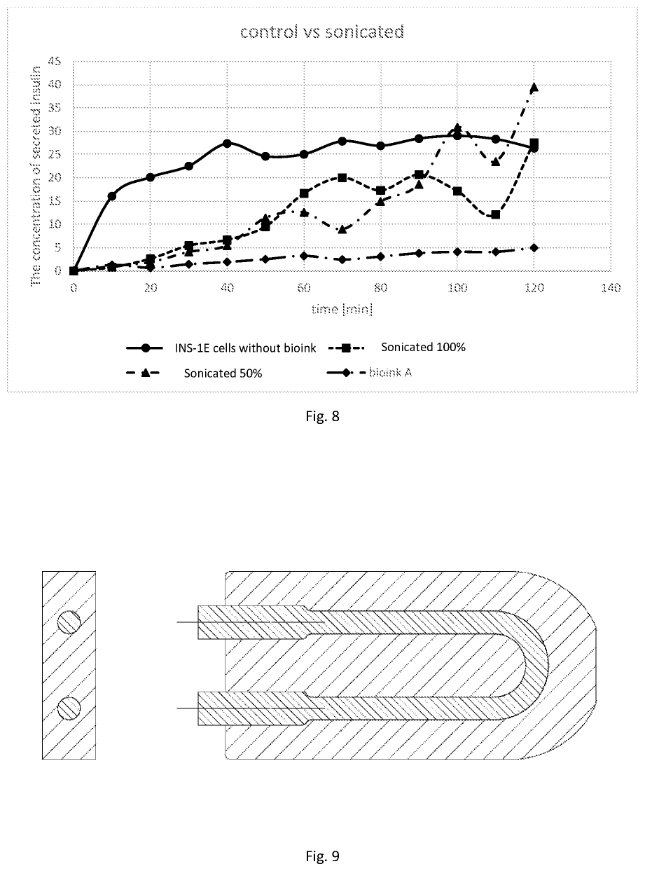

FIG. 8 presents a graph comparing the concentration of secreted insulin in bio-constructs bioprinted with various dECM-based bioinks relative to the control group, meaning 2D cultivation of INS-1E cells.

FIG. 9 presents a schematic drawing of a bioprinted organ with a vessel with a circular cross-section

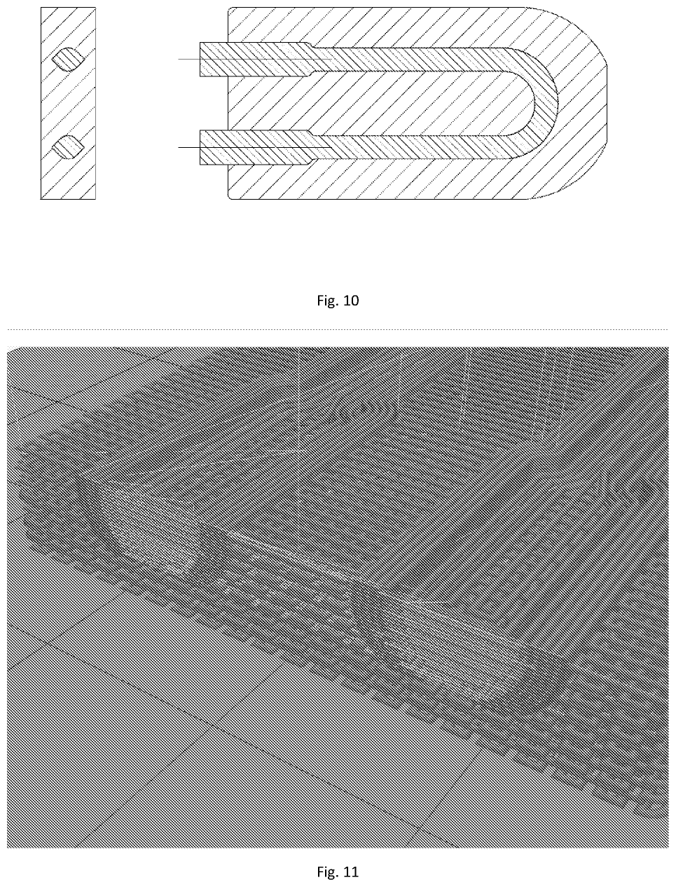

FIG. 10 presents a schematic drawing of a bioprinted organ with a vessel with a circular cross-section of the channel, the same as the cross-section of native vessels present in a living organism.

FIG. 11 presents a drawing presenting the layers of the actual printed pancreas body, the channel layers, and the channel filling.

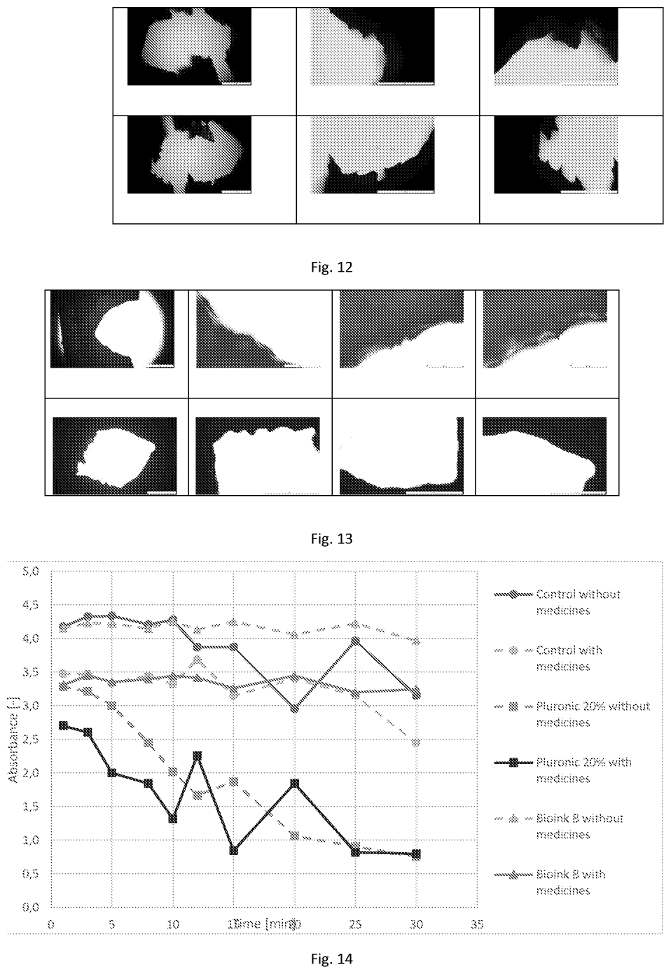

FIG. 12 presents a microscopic image of a channel with a circular cross-section: the surface protrusions and roughness are especially visible in the channel in the upper part of the pancreas, while the channel surface in the lower part of the pancreas is smoother.

FIG. 13 presents a microscopic image of a channel with the cross-section of the vascular system (the lumen of the vessel): the surface protrusions and roughness are especially visible in the channel in the upper part of the pancreas, while the channel surface in the lower part of the pancreas is smoother.

FIG. 14 presents a graph of changes in absorbance over time—the course of a haemolysis test.

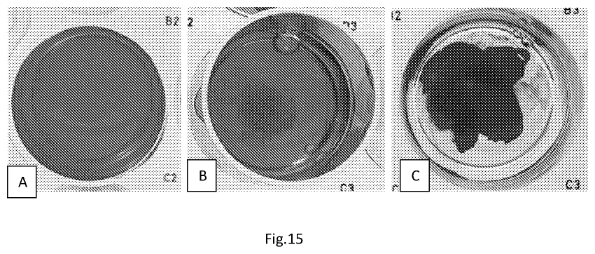

FIG. 15 presents coagulation: A—control, B—bioink B, C—Pluronic.

The model of perFusable printing of a tissue model in 3D bioprinting technology according to the invention allows for printing fibres of the bioink disclosed in application no. PCT/IB2020/056856. The bioink used to produce the model creates structural conditions allowing for the Functioning of living cells not only within it, but also on its surface. Bioink (with cells, organoids, micro-organs including pancreatic islets, or without biological material) constituting material for manufacturing the invention consists of natural components, which as a result form a mixture capable of being used in printing with the extrusive method. In order to maintain the vital functions of the cells contained in the tissue model, apart from its solid structure, the model has in its plan a channel with a diameter within a range of 1.0-3.0 mm over its entire span. The walls closing the channel in its upper part are printed according to two fibre arrangement patterns. In the First one, the Fibres are arranged perpendicularly to the channel axis (FIG. 1A), and in the second pattern they are printed parallel to the channel axis (FIG. 1B). The optimal and the only proper one is printing in the channel along the X axis (FIG. 1B).

Regardless of the adopted fibre arrangement technique, the resulting channel remains tight and unobstructed. These Features enable the Flow of fluids in the lumen of the channel, which can be, e.g. media with compositions corresponding to the cultivation requirements of the cells contained therein (scientific research) or blood after transplanting a bionic organ into the recipient's body. It is because of the blood flow that option B is the only correct solution, since only with such printing technique is the risk of coagulation inside the bionic pancreas significantly reduced, which was proven during tests on large animals (FIG. 2).

The bioprinting method according to the invention uses a number of changes compared to the methods known from prior art. The cross-sectional shape of the channel was changed from circular (FIG. 9) to the same as the cross-section of native vessels present in a living organism (FIG. 10). The change in the printing method for the channel walls involves the use of a plurality of print heads—head no. 1 Filling the organ, head no. 2—the vessel walls, head no. 3 Filling the channel—supporting bioink, and various diameters of the printing needle have been used—a diFFerent diameter for filling the organ, a diFFerent one for printing the channel walls.

Due to the introduction of these changes, the roughness of the upper and lower part of the channel surface is reduced (FIG. 13). A better match of the printing shape was achieved. The printing method according to the invention also results in less errors during printing—elimination of poorly adhering bioink fibres. Improved quality of printing—no need for replacing the bioink cartridge for the head printing the channel walls. Cartridge replacement always interferes with the printing process—changing the temperature of the bioink, mechanically engaging the device—the possibility of losing calibration.

### EMBODIMENTS

Pluronic®, meaning a nonionic copolymer surfactant, was used as supporting bioink.

### Cells:

Red Fluorescent human dermal fibroblasts (HDFa) and green fluorescent human umbilical vein endothelial cells (HUVEC) were used as the model. HDFa cells with ATCC.

### Embodiment 1: Methacrylation of Gelatin in a Carbonate Buffer

1. Place a three-necked flask with a volume of 250-1000 ml provided with a stirring element in a heat block or a water bath (a crystalliser with a volume of 5 l filled with water to half of its volume). By means of a measuring cylinder, measure out 100-500 ml of a 1 M carbonate buffer (1 M sodium bicarbonate and 1 M carbonate in a proportion of 1:1 V/V) and pour into the Flask.

2. Secure the Flask necks with a rubber septum, mount a thermocouple in one of the lateral ones, so that the sensor would be submerged in the solution, but do not hinder mixing, and place a venting needle in the central one. Protect the Flask against light by means of aluminium foil, and activate heating to the preset temperature of 50° C.

3. Weigh out 1-1000 g of gelatin on an analytical balance, and add in small portions to the buffer solution, as a result producing a 10% solution (mass/volume). Leave the mixture constantly stirred (1000 rpm), until complete dissolution of the substrate (approx. 30 min.).

4. After complete dissolution of the substrate, measure the pH of the resulting solution, and write down in a notebook as pH₁.

5. Measure out an amount of anhydride into a syringe with a volume of 1-20 ml provided with a needle, according to table 1 and the intended degree of substitution of methacrylic anhydride, according to the Following procedure: 1) Weigh an empty syringe with a tube and a needle (m₁); 2) measure out the target volume of MMA into the syringe, so that no air bubbles are present in the system, and weigh again (m₂); 3) After adding MMA, weigh the dispensing system again in order to determine the precise amount of MMA added to the reaction mixture (m₃).

TABLE 1
Adjusting the amount of MMA to DS.

| No. | The amount of MMA [ml]/10 g of gelatin | DS [%] |
|---|---|---|
| 1 | 0.5 | ≈40 |
| 2 | 0.6 | ≈50 |
| 3 | 0.8 | ≈60-70 |
| 4 | 1.0 | ≈80 |
| 5 | 1.2 | 100 |

6. Wrap the syringe with MMA in aluminium foil and place on a syringe pump, and stick the needle with the connected tube in the septum, in the lateral neck of the Flask, so that the dispensed anhydride would not flow over the wall, but directly to the reaction mixture. Set a proper flow rate and dispense it until complete depletion of the solution in the syringe, from time to time checking the efficiency of stirring and the viscosity of the reaction mixture. IF necessary, increase the rotational speed of the stirrer.

7. After dripping in the entirety of the measured out MMA, weigh the syringe again and calculate the precise amount of MMA added to the reaction (see pt 5). Continue the reaction under the initial conditions (T=50° C., mixing at 1000-1200 RPM) for approx. 1 h.

8. After this time, measure the pH of the post-reaction mixture by means of a pH meter (write down as pH₂) (see pt 4).

9. Subsequently, add the PBS×1 solution in portions (400-2000 ml) to the mixture, until achieving 5-time dilution oF the reaction mixture (1:4 mixture:PBS×1).

10. Pour the resulting solution into previously prepared dialysis tubes according to the Following procedure:

11. Perform the dialysis process in a temperature of 40° C. For 3 days, replacing water 2 times a day (usually at 08:00 and 16:00). A total of 6 pourings.

12. After completing the dialysis process, transfer the solution from the dialysis tubes quantitatively to a large beaker, and subsequently transfer it to a round-bottom flask with a volume of 1000 ml, approx. 600 ml of the solution at a time, and concentrate it on a rotary evaporator to approx. 25-35% of the initial volume (to no less than 150 ml). Final parameters of concentration:
a) bath temperature: 45° C.
b) pressure: 30 mbar
c) revolutions: 150 rpm
d) initial condenser temperature: −2° C.

13. After the completed concentration process, transfer the solution from the Flask, approx. 12-13 ml at a time (up to a maximum of 1 cm in height) by a serological pipette into plastic urine cups with a volume of 100 ml, described according to the Following template: From the top left corner down: 1. sample name (e.g. W1 GELMA), 2. m₁=vessel mass without the lid, 3. m₂=vessel mass with the solution, 4. m₃=vessel mass with the lyophilisate; bottom right corner—sample preparation date; top right corner-initials of the person preparing it.

14. Place the urine cups with the solution in a freezer in −80° C. and freeze them for at least an hour. Subsequently, transport the Frozen samples in an extruded polystyrene foam box for lyophilisation. Lyophilisation parameters:
a) shelf temperature: 0° C.
b) pressure: 0.100 mbar
c) duration: 24-48 h

15. Weigh the resulting lyophilisate; write down the mass on the urine cup (see pt 14), and determine the water content percentage for each sample in order to determine the efficiency of lyophilisation. To this end, enter all mass values in an Excel program file, and use the Following relationship:

$$M {\%}_{H 2 O} = {m}_{3} - {m}_{1} / {m}_{2} - {m}_{1} * 100 \%$$

16. Collect the resulting product in a collective urine cup, rejecting samples in which the water content percentage significantly differs from the others. Protect the container with parafilm, and store it in a temperature of −20° C.

### Embodiment 2: Methacrylation of Hyaluronic Acid

1. Mount a round-bottom flask with a volume of 100-1000 ml provided with a stirring element on a magnetic stirrer and, by means of a measuring cylinder, measure out 100-500 ml of a carbonate buffer thereinto. Start the stirrer and set the speed at approx. 1000 rpm (depending on the size of the Flask and the stirring element).

2. Weigh out 0.5-5 g of sodium hyaluronate on an analytical balance, and add in small portions to the Flask with the buffer, so that a 1% solution (mass/volume) would be produced as a result. Leave the mixture constantly stirred (1000-1200 rpm) in room temperature until complete dissolution of the substrate (approx. 1 h).

3. After dissolution of the substrate, wrap the Flask in aluminium foil and place it in a water bath (a crystalliser filled with a saturated NaCl solution and ice to half of its volume, so as to maintain lability of the liquid phase). Expected bath temperature ≤0° C.

TABLE 2
Selecting the proper volume of the reaction vessel.

| | The amount of | Substrate | Flask |
|---|---|---|---|
| No. | the buffer (ml) | mass (g) | size (ml) |
| 1 | 50 | 0.5 | 100 |
| 2 | 100 | 1 | 250 |
| 3 | 500 | 5 | 1000 |

4. Measure the pH of the produced solution, and write down as the pH₁ value.

5. By means of an automatic pipette, dispense MMA in equal portions, so that the total dripping time would not exceed 3 h, and the intervals between the consecutive portions would be no shorter than 15 mins.

TABLE 3
Adjusting the amount of MMA to DS.

| | The amount of MMA (ml)/1 g | |
|---|---|---|
| No. | of hyaluronate | DS |
| 1 | 1 | 5-10% |
| 2 | 2 | 15-20% |

6. After dripping in the entirety of the measured out MMA, continue the reaction under the preset conditions (T≤4° C., mixing at 1000-1200 RPM) for another 1 h; after this time, measure the pH₂. Subsequently, replace the ice and saline in the bath, and continue the reaction for another 24 h.

7. After 24 h measure the pH₃ of the post-reaction mixture, and add 1000 ml of cooled demineralised water (T≤5° C.) thereto in order to achieve double dilution (1:1 post-reaction mixture/water).

TABLE 4
A sample list of applied reagents

| No. | Name | CAS no. | Amount | |
|---|---|---|---|---|
| 1 | Sodium hyaluronate Lab. | CAS: 9067-32-7 | 1.00 | g |
| | Grade, 130-300 kDa | | | |
| 2 | Methacrylic anhydride, | CAS: 760-93-0 | 1.00 | ml |
| | 276685-100 ml, | | | |
| 3 | Carbonate buffer X M CB | CAS: 7542-12-3 | 100 | ml |
| | (NaHCO₃:Na₂CO₃ 1:1) | 7542-12-3 | | |
| 4 | Demineralised water | | 100 | ml |

8. Pour the resulting solution into dialysis tubes according to the Following procedure:

9. Perform the dialysis process in a temperature of 30° C. For 4 days, replacing water 2 times a day.

10. After the completed dialysis process, transfer the solution from the Flask, approx. 12-13 ml at a time (up to a maximum of 1 cm in height) by a serological pipette into plastic urine cups with a volume of 100 ml described according to the Following template: From the top left corner down: 1. sample name (e.g. T10 HAMA), 2. m₁=vessel mass without the lid, 3. m₂=vessel mass with the solution, 4. m₃=vessel mass with the lyophilisate; bottom right corner-sample preparation date; top right corner-initials of the person preparing it.

11. Place the urine cups with the solution in a freezer in −80° C. and freeze them for at least an hour. Subsequently, transport the Frozen samples for lyophilisation. Lyophilisation parameters:
d) shelf temperature: −5-0° C.
e) pressure: 0.100-0.500 mbar
F) duration: 24-48 h

12. Weigh the resulting lyophilisate; write down the mass on the urine cup (see pt 10), and determine the water content percentage for each sample in order to determine the efficiency of lyophilisation. To this end, enter all mass values in an Excel program file, and use the Following relationship:

$$M {\%}_{H 2 O} = {m}_{3} - {m}_{1} / {m}_{2} - {m}_{1} * 100 \%$$

13. Collect the resulting product in a collective urine cup, rejecting samples in which the water content percentage significantly differs from the others. Protect the container with parafilm, and store it in a temperature of −20° C.

### Embodiment 3: Preparation of Bioink—the General Procedure

The decellularization material was acquired from a local slaughterhouse, and directly after preparation, the pancreas tissues were precisely cleaned of fat, large vessels and connective tissue, and stored in a PBS solution prior to further handling. The entire process was conducted in accordance with the protocol published by: Klak M, Int J Mol Sci 2021; 22:1-16. https://doi.org/10.3390/ijms22137005.

### Embodiment 4: Bioink a

The powdered form of dECM was dissolved in pepsin (Sigma-Aldrich), and gelatin methacrylate (GelMA; Polbionica Ltd.) or gelatin methacrylamide and hyaluronic acid methacrylate (HAMA; Polbionia Ltd.) were used as the crosslinking agent. Lithium phenyl-2,4,6-trimethylbenzoylphosphinate (LAP) was used as the photoinitiator in UV-vis light. The mixture was used for extrusive bioprinting.

### Embodiment 5: Bioink B

dECM was dissolved in 1×PBS and stirred for 5 minutes at 400 rpm. Subsequently, a thermocouple was connected to a Bandelin Sonopuls HD 3100 ultrasonic homogeniser (Bandelin electronic GmbH & Co. KG, Berlin, Germany) in order to control the temperature oF the sample during sonication. The hydrogel sample was placed on ice and treated with a sonotrode: an MS72 probe with an amplitude of 10-65%. The process was performed in a temperature never exceeding 37° C. For a time of 3-5 minutes.

### Embodiment 6: Printing the Tissue Model—the General Procedure

The tissue model was printed with the extrusive method, in which the temperature of the head with the material used for printing was 18-26° C. The printing needle had a diameter of 609 nm. The pressure inside the needle oscillated within a range of 5-40 kPa, and the Fibre printing rate was 5-30/s, the length of a single fibre ranging from 150 mm to 5000 mm.

### Embodiment 7: Implantation of Cells in the Process of Printing with Bioink B—Indirect Introduction

Tests were performed in order to verify the biocompatibility of the material. One of the key factors was to assess the printing technique, and its impact on the adhesion of the cells. To this end, human cell line models were used (endothelial cells—HUVEC; Fibroblasts—aHDF in a ratio of 1:2 and a total number of 5-10 million/ml, most preferably: 8 million/ml), constituting precursors in the creation of microvasculature de novo. The cells were introduced into the channels in two ways. While printing the described invention, the cells in a suspension of the so-called vascular bioink (indirect introduction) were fed by way of extrusion, thus filling the channel. The printing of vascular bioink took place when setting the head temperature within a range of 15-25° C. The pressure during printing was 5-30 kPa, and the Fibre production proceeded at a rate of 5-40 mm/s. After the bioprinting process, the system was placed in an incubator in order to increase the temperatures to 37° C. and thus melt bioink B, in order to wash it out and achieve an unobstructed flow system. The removal of bioink proceeds by rinsing the channel with a solution of phosphate-buffered physiological saline, a cell medium or another fluid with properly adjusted temperature—in the case of bioink B—25-37° C. Phosphate-buffered physiological saline may be used to rinse the channel in order to unobstruct it. IF we already have a complete organ with pancreatic cells/islets, we must use properly supplemented fluid in order to provide them with proper nutrients; hence the term: cell medium or fluid.

### Embodiment 8: Implantation of Cells in the Process of Printing by Means of Pluronic® Supporting Bioink-Direct Introduction

Pluronic®, which during further printing of the tissue model served a supporting function, was added into the channel placed inside the tissue model. Once the printing was completed, the tissue model was placed in a temperature oF 2-8° C., in which Pluronic® melts. Then, in the tissue model, 1×PBS was run through the channel (at a preferred rate of 5 ml/min.) in order to wash out Pluronic®. Subsequently, cells in a medium were introduced into the unobstructed channel (direct introduction).

Pluronic® may be also removed by rinsing the channel with a cell medium or another fluid with properly adjusted temperature—in the case of Pluronic®—4-8° C. Phosphate-buffered physiological saline may be used to rinse the channel in order to unobstruct it. IF we already have a complete organ with pancreatic cells/islets, we must use properly supplemented fluid in order to provide them with proper nutrients; hence the term: cell medium or fluid.

### Embodiment 9: Testing Tissue Models Printed with Bioink B and Pluronic Supporting Bioink

In the case of both the Former and the latter cell introduction method, the same procedure was used, which involved placing the tissue model in a bioreactor/incubator, in particular such as described in PCT/IB2021/062190, with conditions established as 37° C., and a CO₂ content amounting to 5%. The respective incubation times of the tissue model along with HUVEC and aHDF cells were set as: 2 h, 5 h, 8 h, and 24 h. Once the time of the tested options of incubation has passed, the channel was rinsed with 1×PBS at a preferred rate of 5 m₁/min. For 1-10 minutes (optimally, 2 minutes). The abovementioned tests were performed for tissue models printed using both methods of arranging the Fibre Forming the channel. Three replications were performed for each of the presented versions. A phenomenon of the adhesion of the introduced endothelial cells and fibroblasts was observed regardless of the selected channel printing technique. The cells exhibited morphological changes in the Form of flattening, indicating cultivation conditions favouring growth and proliferation. The number of cells washed out from the channels, expressed in percentage points for the tested options of incubation time, and taking into account the cell introduction method, is presented in FIG. 3.

FIG. 4 presents the longevity of washed out cells expressed in percentage points for the tested options of incubation time in a bioreactor/incubator, taking into account the printing technique (channel printing in the X and Y axes) and the cell introduction method (indirectly, using the so-called vascular bioink; directly, after washing out Pluronic). The error bars correspond to standard deviations.

An analysis of the presented results allows for choosing both the most preferable printing technique and the method of introducing the cells forming microvascularisation. Five-hour incubation of the tissue model whose channels are printed from Fibres arranged spatially in the Y axis, combined with direct introduction of cells, results in the most preferable ratio of the cells remaining in the channel to the survival rate. In the above option, 51.7% oF cells remain in the channel, and their survival rate is 74% (±7% SD). Cultivation of the cells remaining in the channel was performed in a closed flow system. The medium flowed in the channels of the tissue model at a rate of no less than 0.3 ml/min., which value stimulates the cells to adhere and proliferate. The quantity of cells from the option described above did not drop over 8 days. The cells did not circulate in the closed system; they still remained within the channel. After the completed eight-day experiment, the tissue model was secured by 5% Formaldehyde, and subsequently immunohistochemical haematoxylin and eosin staining was performed in order to verify the presence of cells on the surfaces of the channel walls, having previously prepared photographic documentation of the content of the channel (FIGS. 5A and 5B).

### Embodiment 10: Bioprinting a Bionic Pancreas—(BT1)

Description of the model: Pancreas body printed with bioink A, channel filling printed with bioink B/Pluronic. A pancreas model printed by extrusion printing, using a nozzle with a diameter of 100 μm to 650 μm (pancreas body and channel filling). The height of each of the layers is 0.1-1.2 mm. The layers of the model filled in 10 to 100%, containing a double outline of the pancreas body and the channel—ensuring higher precision and tightness of the printed channel. The pathways of the consecutive layers were arranged at an angle of 90° relative to each other. The channel is placed within the model, meaning under and above the channel there are pancreas body layers. The layers surrounding the channel are printed parallel to each other, always in the same direction.

Printing pattern: Printing the model requires the use of two print heads with temperature control.
The layers opening and closing the pancreas body are in a number of 1-10 layers each, with a height of 0.1-1.2 mm.
The layers of the actual pancreas body (with cells/islets) and the outline of the vascular channel are printed while maintaining the same layer height.
Filling the channel with bioink B or Pluronic proceeds without crosslinking.

### Embodiment 11: Bioprinting a Bionic Pancreas—(BT2) Various Heights of Layers

Description of the model: Pancreas body printed with bioink A, channel filling printed with bioink B and/or Pluronic. A pancreas model printed with the use of two heights of layers. The pancreas body and the channel filling printed with extrusion printing using a nozzle with a diameter oF 100 μm to 650 μm; the outlines oF the channel printed with a 50-500 μm needle. Abiding by the rule that there are 2-4 layers of the pancreas channel outline per 1 layer of pancreas body filling. The layers of the model filled in 10 to 100%; the pathways of the consecutive layers arranged at an angle of 90° relative to each other. The channel is placed within the model, meaning under and above the channel there are pancreas body layers. The layers surrounding the channel are printed parallel to each other, always in the same direction.

Printing pattern: Printing the model requires the use of three print heads with temperature control.
The layers opening and closing the pancreas body are in a number of 1-10 layers each, with a height of 0.1-1.2 mm.
The layers of the actual pancreas body (with cells/islets), printed in a sandwich system with a layer height ratio of 1:1.1 to 1:2.0 relative to the outline of the channel (the Flow system).

### Embodiment 12: Description of the Geometry of the Printed Organ (Model), and a Functional Assessment

The axes of the vessel are in a single plane. It is a plane parallel to the planes of the printed layers of the organ. The cross-section of the vessel has the same shape as the cross-section of native vessels present in a living organism, so as to ensure the best possible surface smoothness of the vessels produced in the 3D printing technology.

Functional tests were performed for beta INS-1E cells. To this end, pieces were printed from the 4 main types of bioink, in which the central part was filled with cells. Bioinks of a given type were diversified in terms of the amount of dECM powder.

Varieties of dECM-based bioinks:
A) sonicated 5% hydrogel, and a powder content of 0 to 50%
B) sonicated 10% hydrogel, and a powder content of 0 to 50%
C) nonsonicated 5% hydrogel (prepared by enzymatic digestion), and a powder content of 0 to 50%
D) nonsonicated 10% hydrogel (prepared by enzymatic digestion), and a powder content of 0 to 50%

The Functionality of beta cells was proven in each bioink; however, after 7 days of observation (cultivation in 37° C.; 5% CO₂) and the performance of glucose stimulation tests, it was proven that the best longevity was observed for sonicated dECM-based bioink with a dECM content ranging from 1% to 50%. The First diffusion signal was observed in the 2nd-3rd min. of the experiment (the graph in FIG. 10).

### Embodiment 13: Coagulation Tests—the Course of a Haemolysis Test

The First step of the experiment involved pouring sterile biomaterials (dECM-based bioink (including bioink B) and Pluronic) (200 μl) on the surface of pits (with a diameter of 16 mm) in a 24-pit plate.

Subsequently, Fresh whole blood without an anticoagulant was collected from large animals which had been receiving anticoagulants, and from animals which had not received these medications. 200 μl of whole blood were applied on each of the tested biomaterials.

After the incubation time, 1000 μl of deionised water were added to the samples (so as to cover the entire tested sample). Subsequently, the plate was shaken for 30 seconds, 100-300 rpm.

The next step involved the incubation of the plate for 5 minutes motionlessly, in order to release free haemoglobin. After this time, 200 μl of the samples were collected in order to read the absorbance at a wavelength of 540 nm.

A positive control was also prepared (which was characterised by maximum haemolysis) for each blood type, along with controls for water and the tested materials (FIG. 15).

The absorbance value is proportional to the concentration of free haemoglobin in deionised water due to the lysis of red blood cells. This method provides indirect correlation with the degree of blood clotting on the surface of the biomaterial (FIG. 14).

A higher absorbance value indicates a higher concentration of haemoglobin, which means less blood clotting on the surface oF the biomaterial.

1-16. (canceled)

17. A method for manufacturing a perfusable three-dimensional tissue model, containing therein a channel distributed across its entire structure, enabling the flow of fluids, containing following steps:
i) Bioprinting a vascular system with the extrusive method wherein bioink for printing a vessel is a mixture comprising nonionic copolymer surfactant and hydrogel containing an extracellular matrix and endothelial cells and fibroblasts,
Wherein the fibres of bioink which form the walls opening and closing the channel in its upper part being printed parallel to the channel axis, and
Bioprinting a model body with the extrusive method wherein bioink for printing a body containing a solution of dECM in pepsin, and
Wherein printing a vessel bioink in fibres which are surrounded by fibres of the body bioink printed in parallel position and are printing in the same direction, and
wherein the temperature of the print head with bioink ranges from 10 to 26° C., the printing needle has a diameter ranging from 100 to 609 nm, the pressure used in the bioprinting process encompasses a range of 5 to 200 kPa, and the fibre printing rate ranges from 5 to 40 mm/s, the length of an individual printed fibre ranging from 150 mm to 5000 mm.
ii) placing the resulting system in an incubator, in a temperature in which bioink for printing the vascular system undergoes melt,
iii) rinsing the channel with a solution of phosphate-buffered physiological saline
iv) optionally, causing growth in the channel by means of cells in a medium,
wherein, the cross-section of the channel being the same as the cross-section of native vessels present in a living organism.

18. The method according to claim 17, characterized in that, the pressure used in the process of bioprinting the body of the model and the layers surrounding the channel ranges from 5 to 40 kPa in the case of printing by means of bioink containing pancreatic islets, and from 5 to 200 kPa in the case of printing by means of bioink containing cells, in particular endothelial cells or fibroblasts.

19. The method according to claim 17, characterized in that, the pressure used in the process of bioprinting a vascular system ranges from 5 to 200 kPa in the case of printing by means of bioink containing cells, in particular endothelial cells or fibroblasts, and at least 5 kPa in the case of printing by means of bioink not containing cells.

20. The method according to claim 17, characterized in that, at least one print head with temperature control is used for printing.

21. The method according to claim 17, characterized in that, bioink for printing the vessels is a composition of hydrogel containing an extracellular matrix, with a concentration of 5 to 10% in a solution of phosphate-buffered physiological saline, produced via sonification and endothelial cells and fibroblasts and a compound with the formula (C₃H₆O·C₂H₄O)ₓ, x standing for 10 to 1000 repetitions.

22. The method according to claim 17, characterized in that, the bioink used for printing vessels contains a suspension containing endothelial cells and fibroblasts in a ratio of 1:2, and with a total number of cells of 5 to 10 million/ml, preferably with the number of cells being 8 million/ml in hydrogel containing an extracellular matrix with a concentration of 5 to 10% in a solution of phosphate-buffered physiological saline.

23. The method according to claim 17, characterized in that, the bioink used for printing the body contains at least one crosslinking agent and a photoinitiator; preferably, the crosslinking agent is gelatin methacrylate, gelatin methacrylamide and/or hyaluronic acid methacrylate, and the photoinitiator is lithium phenyl-2,4,6-trimethylbenzoylphosphinate.

24. The method according to claim 17, characterized in that, step ii) takes place in a temperature of no more than 37° C., over a time of no more than 24 h, preferably over a time of 30 to 40 min.

25. The method according to claim 17, characterized in that, the cells for growing in step iv) are selected from a group of: endothelial cells, fibroblasts or a mixture thereof in proportions of 1:2; preferably, the cells grown in the channel have a concentration of 5 to 10 million/ml, preferably 8 million/ml in a culture medium.

26. A bionic model with a perusable system, obtainable by the process of claim 17, characterized in that:
there are 2 to 4 outline layers for the bionic channel of the model per each model body filling layer, the pathways of the consecutive layers being arranged at an angle up to and including 900 relative to each other, and the layers surrounding the channel are parallel to each other, and are in the same direction, and
wherein the layers opening and closing the model body, and the layers opening and closing the vascular channel are in a number of 1 to 10 layers; each layer has a height of 0.1 to 1.2 mm; and
wherein the layers of the body are in a sandwich system with a ratio of the height of the layers to the height of the channel outline wall ranging from 1:1.1 to 1:2.

27. The bionic model according to claim 10, characterized in that, the bionic model has a channel distributed across its entire structure, the cross-section of the channel being the same as the cross-section of native vessels present in a living organism.
